(19) United States
(12) Patent Application Publication
(10) Pub. No.: US 2018/0193056 A1
(43) Pub. Date: Jul. 12, 2018

(54) ATHERECTOMY MEDICAL DEVICE

(71) Applicant: BOSTON SCIENTIFIC SCIMED, INC., MAPLE GROVE, MN (US)

(72) Inventors: CALEB SCOTT COLYER, ST. MICHAEL, MN (US); DAVID GORDON SPANGLER, WHITE BEAR LAKE, WI (US); JASON T. ANDERSON, DEEPHAVEN, MN (US); DANIEL FRANK MASSIMINI, MAPLE GROVE, MN (US); DAVID B. MORRIS, ANOKA, MN (US); LASZLO TRENT FARAGO, HUDSON, WI (US); JOEL R. BREY, SHOREVIEW, MN (US)

(73) Assignee: BOSTON SCIENTIFIC SCIMED, INC., MAPLE GROVE, MN (US)

(21) Appl. No.: 15/870,261

(22) Filed: Jan. 12, 2018

Related U.S. Application Data
(60) Provisional application No. 62/445,562, filed on Jan. 12, 2017.

(51) Int. Cl. A61B 17/3207

(52) U.S. Cl. CPC A61B 17/320758; A61B 2017/320004

(57) ABSTRACT

Medical devices and methods for using medical devices are disclosed. A rotational atherectomy device may include an elongate shaft having a proximal end region, a distal end region and a lumen extending therein. The medical device may also include a cutting member positioned adjacent the distal end region of the elongate shaft. The cutting member may include a plurality of helical grooves defined in an outer surface of the cutting member. At least some of the helical grooves may extend at least one full helical rotation about the cutting member. In some cases, at least some of the helical grooves may include a first helical groove extending in a first direction and a second helical groove extending in a second direction that is different from the first direction.

### CROSS-REFERENCE TO RELATED APPLICATIONS

This application claims the benefit of priority under 35 U.S.C. § 119 to U.S. Provisional Application Ser. No. 62/445,562, filed Jan. 12, 2017, the entirety of which is incorporated herein by reference.

### TECHNICAL FIELD

The present disclosure pertains to medical devices, and methods for manufacturing and using medical devices. More particularly, the disclosure is directed to devices and methods for removing occlusive material from a body lumen. Further, the disclosure is directed to an atherectomy device for forming a passageway through an occlusion of a body lumen, such as a blood vessel.

### BACKGROUND

Many patients suffer from occluded arteries and other blood vessels which restrict blood flow. Occlusions can be partial occlusions that reduce blood flow through the occluded portion of a blood vessel or total occlusions (e.g., chronic total occlusions) that substantially block blood flow through the occluded blood vessel. In some cases a stent may be placed in the area of a treated occlusion. However, restenosis may occur in the stent, further occluding the vessel and restricting blood flow. Revascularization techniques include using a variety of devices to pass through the occlusion to create or enlarge an opening through the occlusion. Atherectomy is one technique in which a catheter having a cutting element thereon is advanced through the occlusion to form or enlarge a pathway through the occlusion. A need remains for alternative atherectomy devices to facilitate crossing an occlusion.

### SUMMARY

This disclosure provides design, material, manufacturing method, and use alternatives for medical devices. For example, the disclosure is directed to an atherectomy device that includes an elongate shaft having a proximal end region and a distal end region and a cutting member that is positioned adjacent the distal end region of the elongate shaft. The cutting member extends along a longitudinal axis and has an outer surface, with a plurality of helical grooves defined in the outer surface of the cutting member, at least two of the plurality of helical grooves extending at least one full helical revolution around the longitudinal axis.

Alternatively or additionally to any of the embodiments above, the plurality of helical grooves may include at least a first groove extending in a first helical direction around the longitudinal axis, and at least a second groove extending in a second helical direction around the longitudinal axis, wherein the first direction is different from the second direction, such that the first groove and the second groove intersect each other at least once.

Alternatively or additionally to any of the embodiments above, the plurality of helical grooves may include first and second grooves extending in a first helical direction around the cutting member.

Alternatively or additionally to any of the embodiments above, the first helical direction is a clockwise direction.

Alternatively or additionally to any of the embodiments above, the first helical direction is a counter clockwise direction.

Alternatively or additionally to any of the embodiments above, at least two of the plurality of grooves may be parallel to one another.

Alternatively or additionally to any of the embodiments above, a helix angle of at least one of the plurality of helical grooves changes as the at least one helical groove extends around the longitudinal axis.

Alternatively or additionally to any of the embodiments above, the helix angle of the at least one helical groove changes in a stepwise fashion.

Alternatively or additionally to any of the embodiments above, the helix angle of the at least one helical groove is continuously changing.

Alternatively or additionally to any of the embodiments above, the plurality of helical grooves have a helix angle in the range of 30 to 60 degrees.

Alternatively or additionally to any of the embodiments above, the plurality of helical grooves includes three or more helical grooves.

Alternatively or additionally to any of the embodiments above, the plurality of helical grooves includes four or more helical grooves.

Alternatively or additionally to any of the embodiments above, the atherectomy device may further include an abrasive material disposed on the outer surface of the cutting member.

Alternatively or additionally to any of the embodiments above, the plurality of helical grooves are substantially free of an abrasive material.

Alternatively or additionally to any of the embodiments above, the plurality of grooves are spaced equally around a central longitudinal axis of the cutting member.

Alternatively or additionally to any of the embodiments above, wherein the atherectomy device further includes a guidewire lumen extending along the longitudinal axis.

The disclosure is also directed to an atherectomy device including an elongate shaft having a proximal end region and a distal end region and a cutting member that is positioned adjacent the distal end region of the elongate shaft. The cutting member extends along a longitudinal axis and has an outer surface with a plurality of helical grooves including at least a first helical groove extending in a first helical direction around the longitudinal axis, and at least a second helical groove extending in a second helical direction around the longitudinal axis, wherein the first direction is different from the second direction, such that the first helical groove and the second helical groove intersect each other at least once.

Alternatively or additionally to any of the embodiments above, the first helical direction is a clockwise direction, and the second helical direction is a counter clockwise direction.

Alternatively or additionally to any of the embodiments above, the atherectomy device further includes one or more additional grooves extending in the first helical direction.

Alternatively or additionally to any of the embodiments above, the atherectomy device further includes one or more additional grooves extending in the second helical direction.

Alternatively or additionally to any of the embodiments above, a helix angle of at least one of the plurality of helical grooves may change as the at least one helical groove extends around the longitudinal axis.

Alternatively or additionally to any of the embodiments above, the helix angle of the at least one helical groove may change in a stepwise fashion.

Alternatively or additionally to any of the embodiments above, the helix angle of the at least one helical groove may be continuously changing.

Alternatively or additionally to any of the embodiments above, the plurality of helical grooves may have a helix angle in the range of 30 to 60 degrees.

Alternatively or additionally to any of the embodiments above, the plurality of helical grooves may include three or more helical grooves.

Alternatively or additionally to any of the embodiments above, the plurality of helical grooves may include four or more helical grooves.

Alternatively or additionally to any of the embodiments above, the atherectomy device may further include an abrasive material disposed on the outer surface of the cutting member.

Alternatively or additionally to any of the embodiments above, the plurality of helical grooves may be substantially free of an abrasive material.

Alternatively or additionally to any of the embodiments above, the plurality of grooves may include two or more helical grooves extending in the first helical direction around the longitudinal axis, and two or more helical grooves extending in the second helical direction around the longitudinal axis.

Alternatively or additionally to any of the embodiments above, the two or more helical grooves extending in the first helical direction around the longitudinal axis are spaced equally around a central longitudinal axis of the cutting member.

Alternatively or additionally to any of the embodiments above, the two or more helical grooves extending in the second helical direction around the longitudinal axis are spaced equally around a central longitudinal axis of the cutting member.

Alternatively or additionally to any of the embodiments above, the atherectomy device further includes a guidewire lumen extending along the longitudinal axis.

The disclosure is also directed to an atherectomy device including an elongate shaft having a proximal end region and a distal end region and a cutting member that is positioned adjacent the distal end region of the elongate shaft. The cutting member extends along a longitudinal axis and has an outer surface including a plurality of helical grooves including at least a first helical groove extending in a first helical direction around the longitudinal axis, and at least a second helical groove extending in a second helical direction around the longitudinal axis, wherein the first direction is different from the second direction, such that the first helical groove and the second helical groove intersect each other at least once. The at least the first helical groove and the at least a second helical groove each extend at least one full helical revolution around the longitudinal axis.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an example atherectomy device;

FIG. 2 is a side view of the example atherectomy device of FIG. 1;

FIG. 3 is an end view of the example atherectomy device of FIG. 1;

FIG. 4 is a perspective view of an example atherectomy device;

FIG. 5 is a side view of the example medical atherectomy of FIG. 4;

FIG. 6 is an end view of the example medical atherectomy of FIG. 4;

FIG. 7 is a side view of an example medical atherectomy device;

FIG. 8 is an end view of the example medical atherectomy device of FIG. 7;

FIG. 9 is a side view of an example atherectomy device;

FIG. 10 is a side view of an example atherectomy device;

FIG. 11A is a schematic diagram of an example groove pattern;

FIG. 11B is a schematic diagram of an example groove pattern;

FIG. 11C is a schematic diagram of an example groove pattern;

FIG. 12A is a schematic diagram of an example groove pattern;

FIG. 12B is a schematic diagram of an example groove pattern;

FIG. 13A is a schematic diagram of an example groove pattern;

FIG. 13B is a schematic diagram of an example groove pattern;

FIG. 13C is a schematic diagram of an example groove pattern;

FIG. 14 is a schematic diagram of an example atherectomy system that may utilize any of the atherectomy devices of FIG. 1 through FIG. 10; and

FIG. 15 is a schematic diagram of another exemplary atherectomy system that may utilize any of the atherectomy devices of FIG. 1 through FIG. 10.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Many patients suffer from occluded arteries, other blood vessels, and/or occluded ducts or other body lumens which may restrict bodily fluid (e.g. blood, bile, etc.) flow. Occlusions can be partial occlusions that reduce blood flow through the occluded portion of a blood vessel or total occlusions (e.g., chronic total occlusions) that substantially block blood flow through the occluded blood vessel. Revascularization techniques include using a variety of devices to pass through the occlusion to create or enlarge an opening through the occlusion. Atherectomy is one technique in which a catheter having a cutting element thereon is advanced through the occlusion to form or enlarge a pathway through the occlusion. Ideally, the cutting element excises the occlusion without damaging the surrounding vessel wall and/or a previously implanted stent where restenosis has occurred. However, in some instances the cutting element may be manipulated and/or advanced such that it contacts the vessel wall and/or the stent. Therefore, it may be desirable to utilize materials and/or design an atherectomy device that can excise an occlusion without damaging the surrounding vessel and/or a previously implanted stent where restenosis has occurred. Additionally, it may be desirable that a cutting element be useful in removing hard occlusive material, such as calcified material, as well as softer occlusive material. The methods and systems disclosed herein may be designed to overcome at least some of the limitations of previous atherectomy devices while effectively excising occlusive material. For example, some of the devices and methods disclosed herein may include cutting elements with unique cutting surface geometries and/or designs.

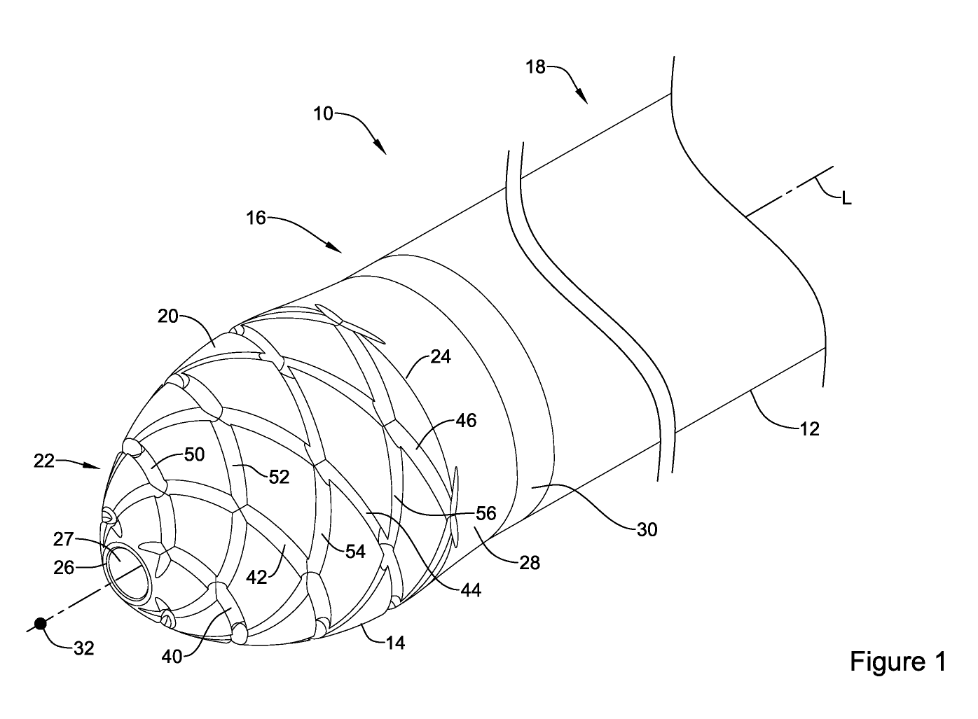

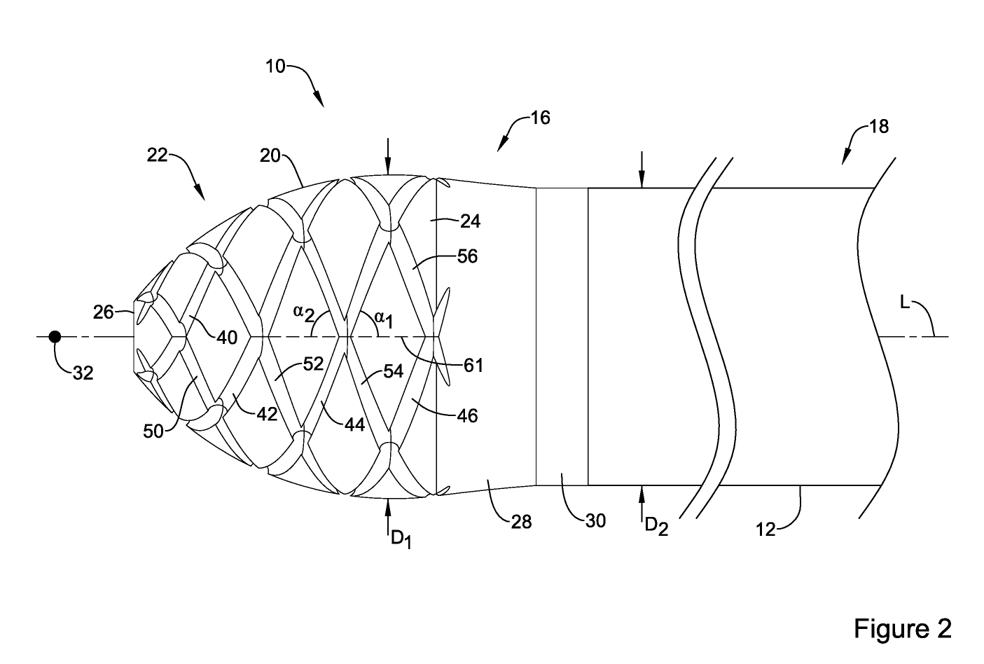

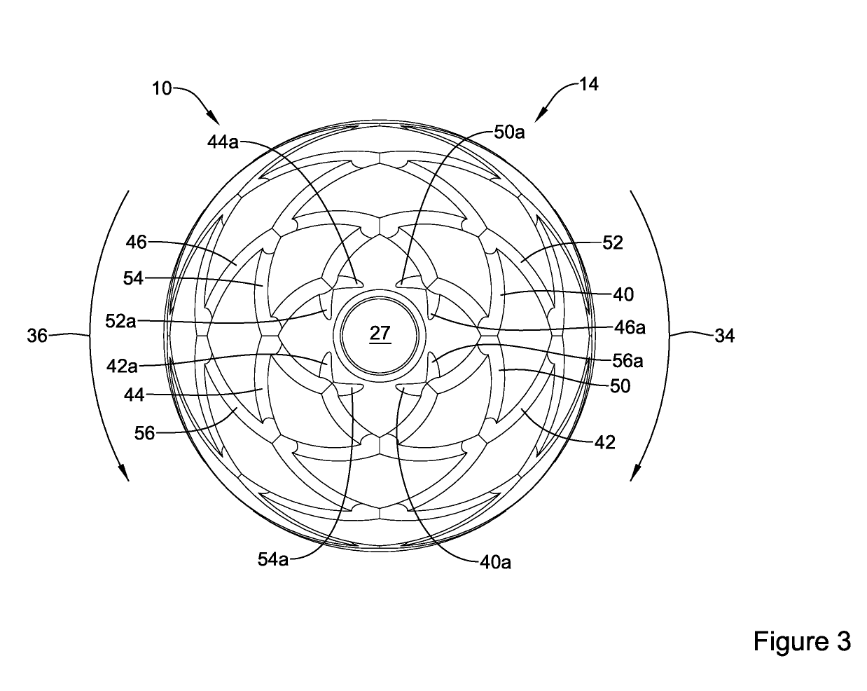

FIG. 1 is a perspective view of an example atherectomy device 10 and FIG. 2 is a side view of the atherectomy device 10. FIG. 3 is an end view of the atherectomy device 10. The atherectomy device 10 includes an elongate shaft 12 and a cutting member 14 that is coupled to the elongate shaft 12. The elongate shaft 12 may be considered as extending from a distal end region 16 to a proximal end region 18. In some cases, as illustrated, the cutting member 14 may be coupled to the distal end region 16 of the elongate shaft 12. The proximal end region 18 is not shown in its entirety, as it will be appreciated that the particular configuration of the proximal end region 18 of the elongate shaft 12 may vary in accordance with the drive mechanism and other features of an atherectomy system with which the atherectomy device 10 may be used. In some cases, a lumen 27 extends through the cutting member 14 and the elongate shaft

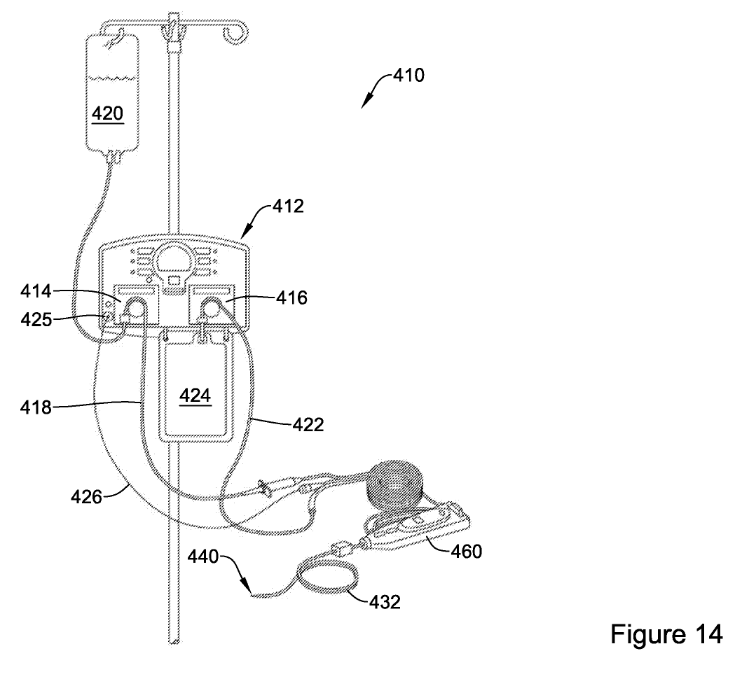

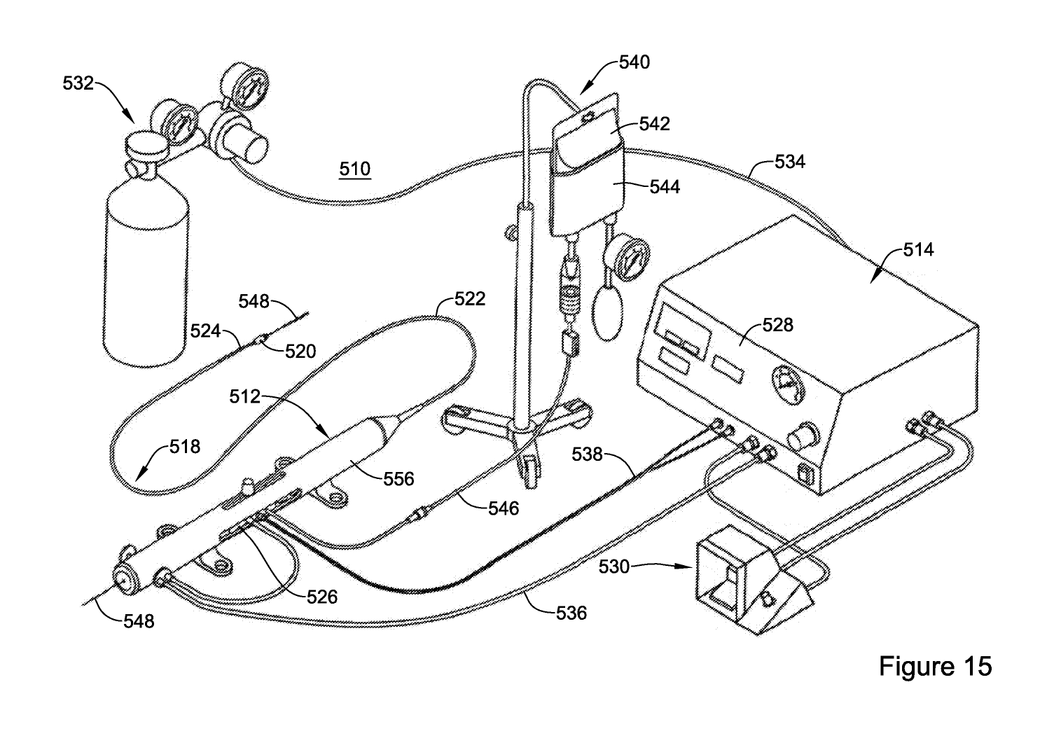

As will be discussed, FIG. 14 illustrates an illustrative but non-limiting atherectomy system with which the atherectomy device 10 may be used. FIG. 15 illustrates another illustrative but non-limiting atherectomy system with which the atherectomy device 10 may be used.

The cutting member 14 has an outer surface 20 and a plurality of grooves 22 defined within the outer surface 20. In some cases, the outer surface 20 is a curved surface, extending from a proximal end 24 of the cutting member 14 to a distal end 26 of the cutting member 14. In some cases, the outer surface 20 curves (relative to the longitudinal axis L) evenly between the proximal end 24 and the distal end 26. In some instances, the outer surface 20 curves at a varying rate that is related to relative position. In other words, in some cases the outer surface 20 curves at a relatively lower rate closer to the proximal end 24 and curves at a relatively higher rate closer to the distal end 26. In some cases, at least some of the plurality of grooves 22 may have a uniform groove profile. In some instances, all of the plurality of grooves 22 may have a uniform groove profile. It will be appreciated that in some of the drawings, some of the plurality of grooves 22 may appear to have a non-uniform groove profile, such as a non-uniform groove width, by virtue of viewing angle, looking at a groove as it extends along a curved surface.

In some cases, the elongate shaft 12 is a unitary member, extending proximally from the cutting member 14. In some instances, the elongate shaft 12 may include one or more intermediate sections, such as but not limited to a tapered section 28 and/or an intermediate section 30 disposed between the cutting member 14 and a remainder of the elongate shaft 12. The tapered section 28 and/or the intermediate section 30 may, for example, be integrally milled or otherwise formed with the remainder of the elongate shaft 12. In some cases, the tapered section 28 and/or the intermediate section 30, if present, may be separately formed and secured to the elongate shaft 12. In some cases, the cutting member 14 may be milled into a blank forming the cutting member 14 and the elongate shaft 12 as a unitary structure. In some instances, the cutting member 14 may be separately formed and subsequently secured to the elongate shaft 12.

In some cases, the outer surface 20 may define an overall diameter of the cutting member 14, indicated for example on FIG. 2 as a diameter D₁, that is greater than an overall diameter of the elongate shaft 12, indicated on FIG. 2 as a diameter D₂. In some cases, providing the elongate shaft 12 with a smaller diameter than that of the cutting member 14 may mean that the cutting member 14 may be dimensioned to fully contact a possible occlusion across a full dimension of the possible occlusion while the elongate shaft 12 may be smaller in diameter than the blood vessel bearing the possible occlusion, thereby reducing potential frictional issues as the elongate shaft 12 rotates. In some instances, the diameter D₁ of the cutting member 14 may be up to 5 percent larger than the diameter D₂ of the elongate shaft 12. In some cases, D₁ may be up to 10 percent larger than D₂, or even larger.

In some cases, the plurality of grooves 22 may include a number of grooves extending in a first helical direction and a number of grooves extending in a second helical direction. In some cases, some of the grooves may extend in a clockwise direction while other grooves extend in a counter-clockwise direction. It will be appreciated that clockwise and counter-clockwise require a vantage point to be defined. For purposes of defining clockwise and counter-clockwise in describing the direction in which grooves may extend, the vantage point is defined as being a point 32 that is distal of the distal end 26 of the cutting member 14 and lies along the longitudinal axis L. From the vantage point 32, and with reference to FIG. 3, it will be appreciated that clockwise may be defined by an arrow 34 and counter-clockwise may be defined by an arrow 36. A groove may be defined as extending in a clockwise direction as the groove extends from a position near the proximal end 24 of the cutting member 14 to a position near the distal end 26 of the cutting member 14.

In some instances, the plurality of grooves 22 may include one, two, three, four, five, six or more distinct grooves extending helically in a first direction and one, two, three, four, five, six or more distinct grooves extending helically in a second direction that is different from the first direction such that the groove(s) extending in the first direction may intersect the groove(s) extending in the second direction. As an illustrative but non-limiting example, in some cases, the cutting member 14 includes four grooves that extend in a clockwise direction and four grooves that extend in a counter-clockwise direction. The four grooves extending in a clockwise direction are labeled as a groove 40, a groove 42, a groove 44 and a groove 46. In some cases, as shown, each of the groove 40, the groove 42, the groove 44 and the groove 46 extend helically all the way around the outer surface 20 of the cutting member 14. The groove 40 may be considered as terminating at a terminus 40a, the groove 42 may be considered as terminating at a terminus 42a, the groove 44 may be considered as terminating at a terminus 44a and the groove 46 may be considered as terminating at a terminus 46a. As shown, each terminus 40a, 42a, 44a and 46a are spaced about 90 degrees apart near the distal end 26 of the cutting member 14.

The four grooves extending in a counter-clockwise direction are labeled as a groove 50, a groove 52, a groove 54 and a groove 56. In some cases, as shown, each of the groove 50, the groove 52, the groove 54 and the groove 56 extend helically all the way around the outer surface 20 of the cutting member 14. The groove 50 may be considered as terminating at a terminus 50a, the groove 52 may be considered as terminating at a terminus 52a, the groove 54 may be considered as terminating at a terminus 54a and the groove 56 may be considered as terminating at a terminus 56a. As shown, each terminus 50a, 52a, 54a and 56a are spaced about 90 degrees apart near the distal end 26 of the cutting member 14.

In some cases, two or more of the groove 40, the groove 42, the groove 44 and the groove 46 may be considered as being parallel to each other. In some cases, two or more of the groove 50, the groove 52, the groove 54 and the groove 56 may be considered as being parallel to each other. As the grooves are defined within a curving surface, in this case parallel may be defined as an axial distance between the two parallel grooves remaining constant as the two parallel grooves extend helically around the outer surface 20.

In some cases, a groove may be considered as having a helix angle, which may be defined in terms of an acute angle formed between the particular groove and a line defined by a plane extending parallel to and through the longitudinal axis L. With reference to FIG. 2, such a reference line 61 is shown. For simplicity, the acute angles formed between a single groove extending in a clockwise direction and a single groove extending in a counter-clockwise direction are shown. As shown, an acute angle α₁ may be defined between the groove 44 and the reference line 61 and an acute angle α₂ may be defined between the groove 54 and the reference line 61. In some cases, α₁ and α₂ may independently range from 30 to 60 degrees. In some cases, α₁ and/or α₂ may remain constant. In some cases, α₁ and/or α₂ may change as the groove extends helically about the cutting member 14. In some cases, α₁ and/or α₂ may change continuously. In some cases, α₁ and/or α₂ may change in a stepwise fashion. In some cases, each of the grooves extending in the clockwise direction and/or each of the grooves extending in the counter-clockwise direction may define a single angle between each of the grooves and the reference line 61. In some cases, one or more of the grooves extending in the clockwise direction and/or one or more of the grooves extending in the counter-clockwise direction may define unique angles between the groove and the reference line 61.

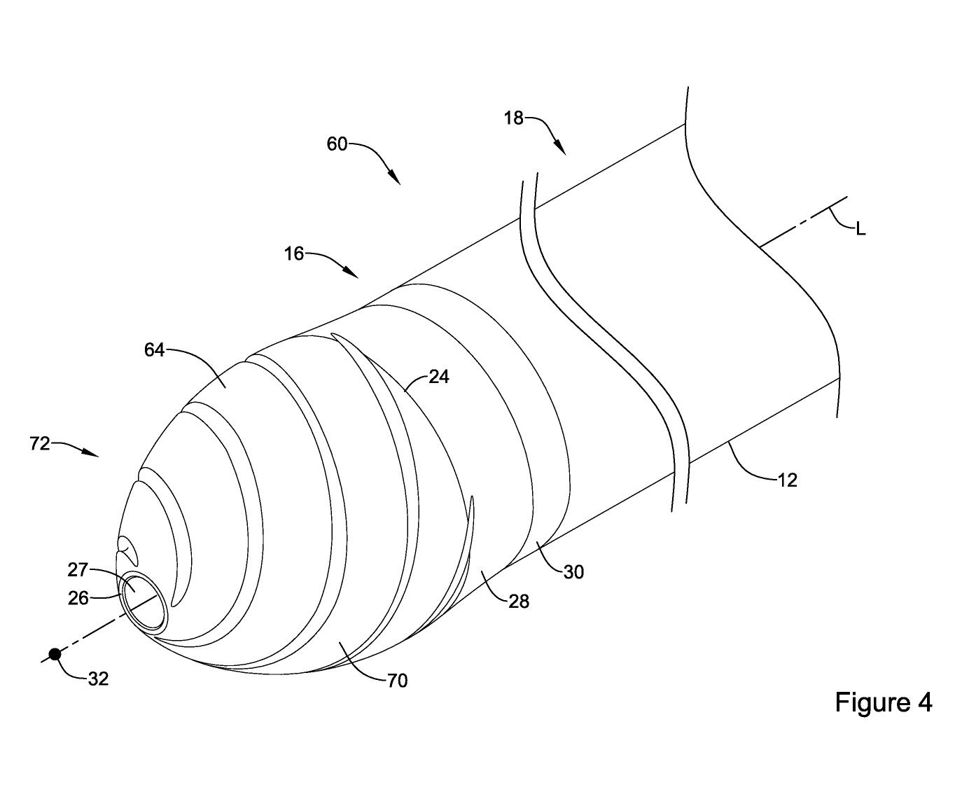

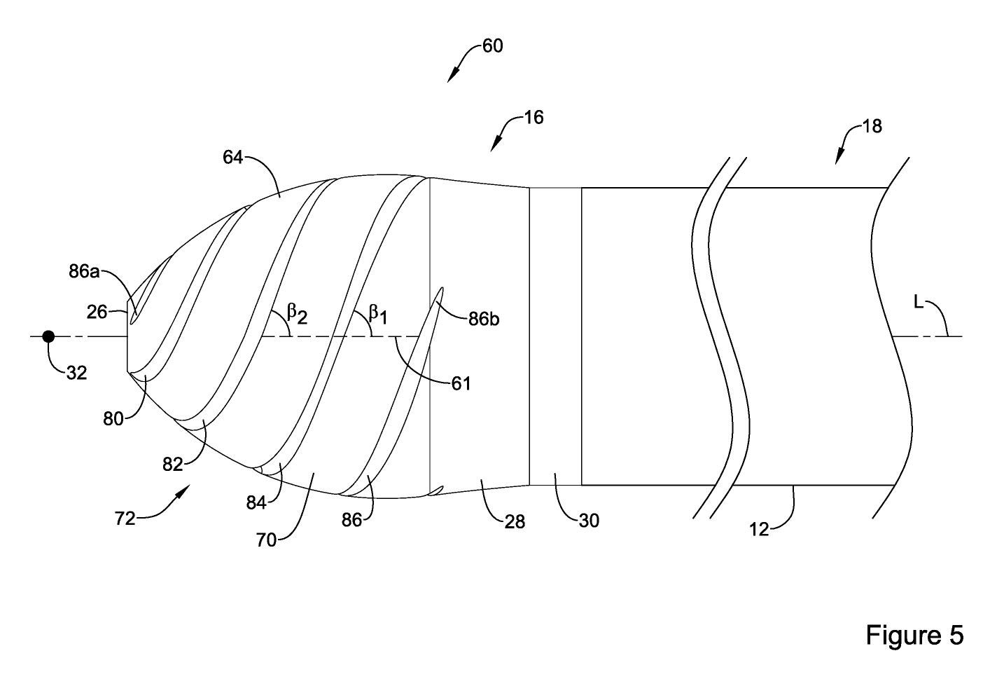

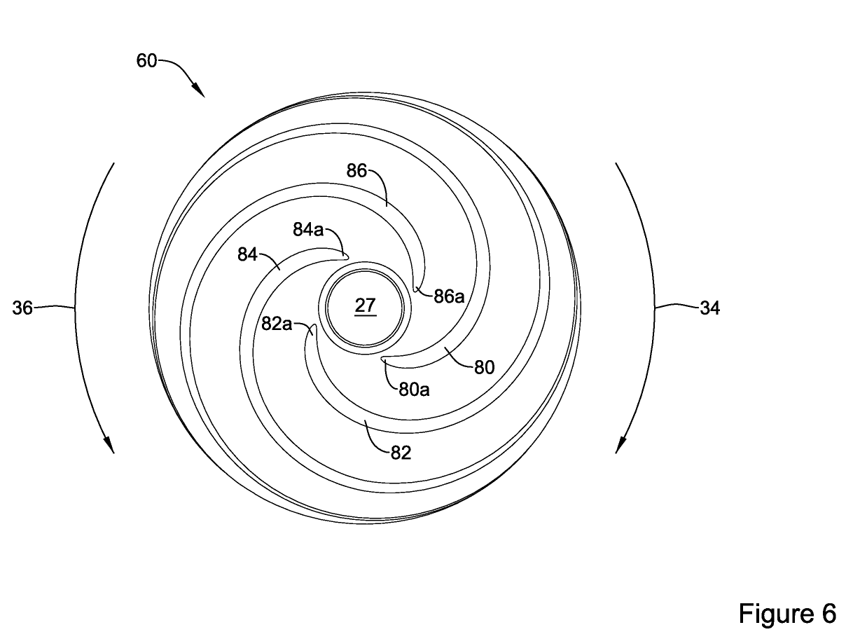

FIG. 4 is a perspective view of an example atherectomy device 60 and FIG. 5 is a side view of the atherectomy device 60. FIG. 6 is an end view of the atherectomy device 60. The atherectomy device 60 includes an elongate shaft 12 and a cutting member 64 that is coupled to the elongate shaft 12. The elongate shaft 12 may be considered as extending from the distal end region 16 to the proximal end region 18. In some cases, as illustrated, the cutting member 64 may be coupled to the distal end region 16 of the elongate shaft 12. The proximal end region 18 is not shown in its entirety, as it will be appreciated that the particular configuration of the proximal end region 18 of the elongate shaft 12 may vary in accordance with the drive mechanism and other features of an atherectomy system with which the atherectomy device 60 may be used.

The cutting member 64 has an outer surface 70 and a plurality of grooves 72 defined within the outer surface 70. In some cases, the outer surface 70 is a curved surface, extending from the proximal end 24 of the cutting member 64 to the distal end 26 of the cutting member 64. In some cases, the outer surface 70 curves (relative to the longitudinal axis L) evenly between the proximal end 24 and the distal end 26. In some instances, the outer surface 70 curves at a varying rate that is related to relative position. In other words, in some cases the outer surface 70 curves at a relatively lower rate closer to the proximal end 24 and curves at a relatively higher rate closer to the distal end 26. In some cases, at least some of the plurality of grooves 72 may have a uniform groove profile. In some instances, all of the plurality of grooves 72 may have a uniform groove profile. It will be appreciated that in some of the drawings, some of the plurality of grooves 72 may appear to have a non-uniform groove profile, such as a non-uniform groove width, by virtue of viewing angle, looking at a groove as it extends along a curved surface.

In some cases, the elongate shaft 12 is a unitary member, extending proximally from the cutting member 14. In some instances, the elongate shaft 12 may include one or more intermediate sections, such as but not limited to the tapered section 28 and/or the intermediate section 30 disposed between the cutting member 64 and a remainder of the elongate shaft 12. The tapered section 28 and/or the intermediate section 30 may, for example, be integrally milled or otherwise formed with the remainder of the elongate shaft 12. In some cases, the tapered section 28 and/or the intermediate section 30, if present, may be separately formed and secured to the elongate shaft 12. In some cases, the cutting member 64 may be milled into a blank forming the cutting member 64 and the elongate shaft 12 as a unitary structure. In some instances, the cutting member 64 may be separately formed and subsequently secured to the elongate shaft 12.

In some cases, the plurality of grooves 72 may include a number of grooves extending in a first helical direction. In some cases, each of the plurality of grooves 72 may extend in a clockwise direction relative to the vantage point 32 that is distal of the distal end 26 of the cutting member 64 and lies along the longitudinal axis L. From the vantage point 32, and with reference to FIG. 6, it will be appreciated that clockwise may be defined by the arrow 34 and counter-clockwise may be defined by the arrow 36. A groove may be defined as extending in a clockwise direction as the groove extends from a position near the proximal end 24 of the cutting member 64 to a position near the distal end 26 of the cutting member 64. In other cases, the plurality of grooves 72 may instead extend in a counter-clockwise direction.

In some instances, the plurality of grooves 72 may include one, two, three, four, five, six or more distinct grooves extending helically in a direction. As an illustrative but non-limiting example, and as shown, the cutting member 64 may include a groove 80, a groove 82, a groove 84 and a groove 86. In some cases, each of the groove 80, the groove 82, the groove 84 and the groove 86 may each extend at least one full revolution about the outer surface 70 of the cutting member 64. This may be seen, for example, in the groove 86, which extends from a first terminus 86a located near the distal end 26 of the cutting member 64 to a second terminus 86b located near the proximal end 24 of the cutting member 64.

With reference to FIG. 6, the groove 80 may be considered as terminating at a terminus 80a, the groove 82 may be considered as terminating at a terminus 82a, the groove 84 may be considered as terminating at a terminus 84a and the groove 86 of course terminates in the terminus 86a. As shown, each terminus 80a, 82a, 84a and 86a are spaced about 90 degrees apart near the distal end 26 of the cutting member 64.

In some cases, two or more of the groove 80, the groove 82, the groove 84 and the groove 86 may be considered as being parallel to each other. As the grooves are defined within a curving surface, in this case parallel may be defined as an axial distance between the two parallel grooves remaining constant as the two parallel grooves extend helically around the outer surface 70.

In some cases, a groove may be considered as having a helix angle, which may be defined in terms of an acute angle formed between the particular groove and a line defined by a plane extending parallel to and through the longitudinal axis L. With reference to FIG. 5, such a reference line 61 is shown. As shown, for example, an acute angle β₁ may be defined between the groove 84 and the reference line 61 and an acute angle β₂ may be defined between the groove 82 and the reference line 61. In some cases, β₁ and β₂ may be equal to each other. In some cases, β₁ and β₂ may independently range from 30 to 60 degrees. In some cases, β₁ and/or β₂ may remain constant. In some cases, α₁ and/or α₂ may change as the groove extends helically about the cutting member 14. In some cases, β₁ and/or β₂ may change continuously. In some cases, β₁ and/or β₂ may change in a stepwise fashion.

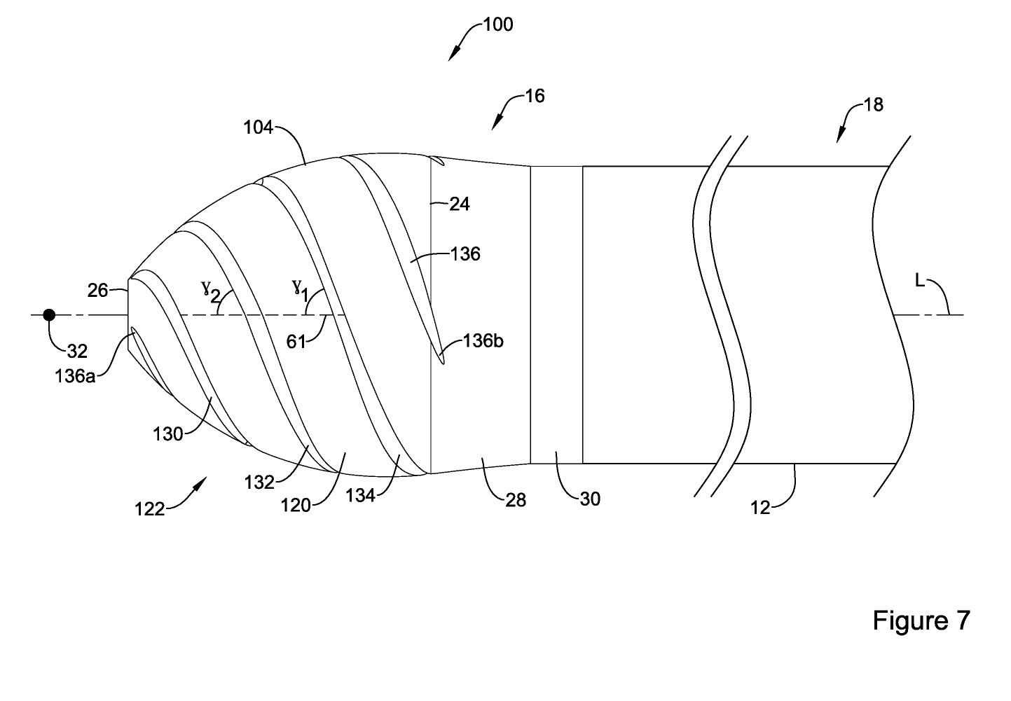

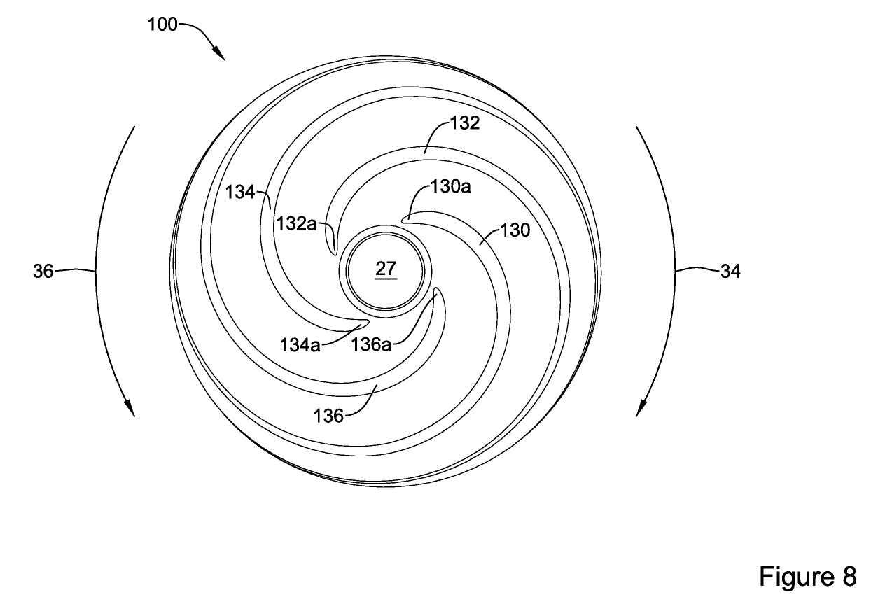

FIG. 4, FIG. 5 and FIG. 6 showed an atherectomy device 60 in which the groove 80, the groove 82, the groove 84 and the groove 86 each extended in a clockwise direction. FIG. 7 is a perspective view of an example atherectomy device 100 having grooves extending in a counterclockwise direction and FIG. 8 is an end view of the atherectomy device 100. The atherectomy device 100 includes an elongate shaft 12 and a cutting member 104 that is coupled to the elongate shaft 12. The elongate shaft 12 may be considered as extending from the distal end region 16 to the proximal end region 18. In some cases, as illustrated, the cutting member 104 may be coupled to the distal end region 16 of the elongate shaft 12. The proximal end region 18 is not shown in its entirety, as it will be appreciated that the particular configuration of the proximal end region 18 of the elongate shaft 12 may vary in accordance with the drive mechanism and other features of an atherectomy system with which the atherectomy device 60 may be used.

The cutting member 104 has an outer surface 120 and a plurality of grooves 122 defined within the outer surface 120. In some cases, the outer surface 120 is a curved surface, extending from the proximal end 24 of the cutting member 104 to the distal end 26 of the cutting member 104. In some cases, the outer surface 120 curves (relative to the longitudinal axis L) evenly between the proximal end 24 and the distal end 26. In some instances, the outer surface 120 curves at a varying rate that is related to relative position. In other words, in some cases the outer surface 120 curves at a relatively lower rate closer to the proximal end 24 and curves at a relatively higher rate closer to the distal end 26. In some cases, at least some of the plurality of grooves 122 may have a uniform groove profile. In some instances, all of the plurality of grooves 122 may have a uniform groove profile. It will be appreciated that in some of the drawings, some of the plurality of grooves 122 may appear to have a non-uniform groove profile, such as a non-uniform groove width, by virtue of viewing angle, looking at a groove as it extends along a curved surface.

In some cases, the elongate shaft 12 is a unitary member, extending proximally from the cutting member 104. In some instances, the elongate shaft 12 may include one or more intermediate sections, such as but not limited to the tapered section 28 and/or the intermediate section 30 disposed between the cutting member 104 and a remainder of the elongate shaft 12. The tapered section 28 and/or the intermediate section 30 may, for example, be integrally milled or otherwise formed with the remainder of the elongate shaft 12. In some cases, the tapered section 28 and/or the intermediate section 30, if present, may be separately formed and secured to the elongate shaft 12. In some cases, the cutting member 104 may be milled into a blank forming the cutting member 104 and the elongate shaft 12 as a unitary structure. In some instances, the cutting member 104 may be separately formed and subsequently secured to the elongate shaft 12.

In some cases, the plurality of grooves 122 may include a number of grooves extending in a helical direction. In some cases, each of the plurality of grooves 122 may extend in a counter-clockwise direction relative to the vantage point 32 that is distal of the distal end 26 of the cutting member 104 and lies along the longitudinal axis L. From the vantage point 32, and with reference to FIG. 8, it will be appreciated that clockwise may be defined by the arrow 34 and counter-clockwise may be defined by the arrow 36. A groove may be defined as extending in a counter-clockwise direction as the groove extends from a position near the proximal end 24 of the cutting member 104 to a position near the distal end 26 of the cutting member 104. In other cases, the plurality of grooves 122 may instead extend in a clockwise direction.

In some instances, the plurality of grooves 122 may include one, two, three, four, five, six or more distinct grooves extending helically in a direction. As an illustrative but non-limiting example, and as shown, the cutting member 104 may include a groove 130, a groove 132, a groove 134 and a groove 136. In some cases, each of the groove 130, the groove 132, the groove 134 and the groove 136 may each extend at least one full revolution about the outer surface 120 of the cutting member 104. This may be seen, for example, in the groove 136, which extends from a first terminus 136a located near the distal end 26 of the cutting member 104 to a second terminus 136b located near the proximal end 24 of the cutting member 64.

With reference to FIG. 8, the groove 130 may be considered as terminating at a terminus 130a, the groove 132 may be considered as terminating at a terminus 132a, the groove 134 may be considered as terminating at a terminus 134a and the groove 136 of course terminates in the terminus 136a. As shown, each terminus 130a, 132a, 134a and 136a are spaced about 90 degrees apart near the distal end 26 of the cutting member 104.

In some cases, two or more of the groove 130, the groove 132, the groove 134 and the groove 136 may be considered as being parallel to each other. As the grooves are defined within a curving surface, in this case parallel may be defined as an axial distance between the two parallel grooves remaining constant as the two parallel grooves extend helically around the outer surface 120.

In some cases, a groove may be considered as having a helix angle, which may be defined in terms of an acute angle formed between the particular groove and a line defined by a plane extending parallel to and through the longitudinal axis L. With reference to FIG. 7, such a reference line 61 is shown. As shown, for example, an acute angle γ₁ may be defined between the groove 134 and the reference line 61 and an acute angle γ₂ may be defined between the groove 132 and the reference line 61. In some cases, γ₁ and γ₂ may be equal to each other. In some cases, γ₁ and γ₂ may independently range from 30 to 60 degrees. In some cases, γ₁ and γ₂ may remain constant. In some cases, α₁ and/or α₂ may change as the groove extends helically about the cutting member 14. In some cases, γ₁ and/or γ₂ may change continuously. In some cases, γ₁ and/or γ₂ may change in a stepwise fashion.

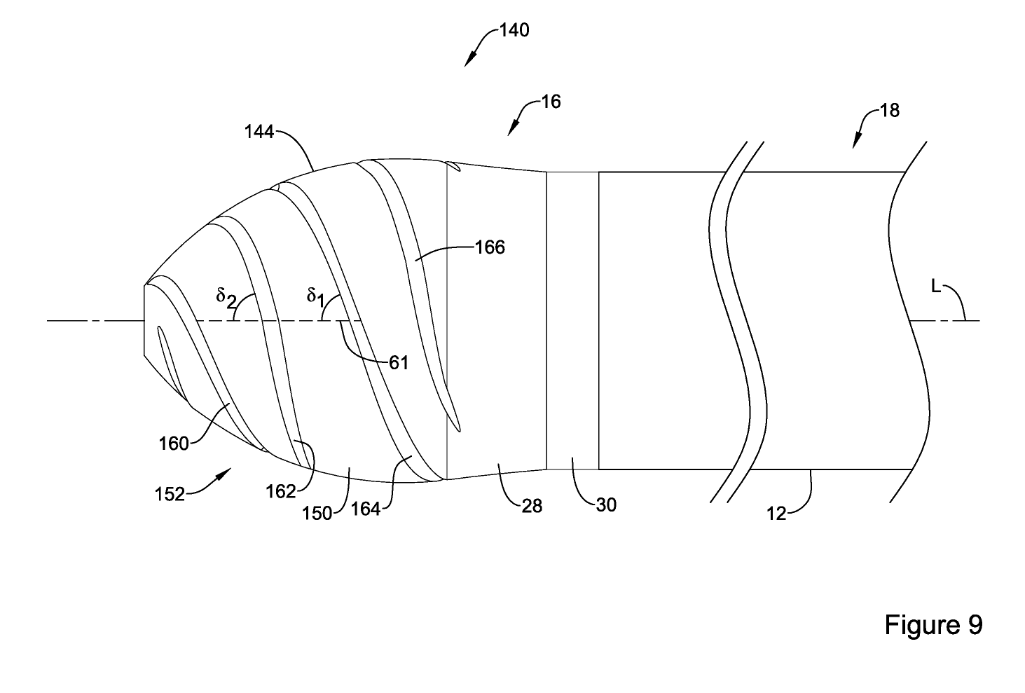

As shown in FIG. 7 and FIG. 8, the atherectomy device 100 includes grooves defining angles γ₁ and γ₂ that remained equal to each other and the same for each groove. In some cases, these angles do not need to remain equal and constant. For example, FIG. 9 is a side view of an example atherectomy device 140 in which the angles vary from groove to groove. The atherectomy device 140 includes an elongate shaft 12 and a cutting member 144 that is coupled to the elongate shaft 12. The elongate shaft 12 may be considered as extending from the distal end region 16 to the proximal end region 18. In some cases, as illustrated, the cutting member 104 may be coupled to the distal end region 16 of the elongate shaft 12. The proximal end region 18 is not shown in its entirety, as it will be appreciated that the particular configuration of the proximal end region 18 of the elongate shaft 12 may vary in accordance with the drive mechanism and other features of an atherectomy system with which the atherectomy device 14 may be used.

The cutting member 144 has an outer surface 150 and a plurality of grooves 152 defined within the outer surface 150. In some cases, the outer surface 150 is a curved surface, extending from the proximal end 24 of the cutting member 144 to the distal end 26 of the cutting member 144. In some cases, the outer surface 150 curves (relative to the longitudinal axis L) evenly between the proximal end 24 and the distal end 26. In some instances, the outer surface 150 curves at a varying rate that is related to relative position. In other words, in some cases the outer surface 150 curves at a relatively lower rate closer to the proximal end 24 and curves at a relatively higher rate closer to the distal end 26. In some cases, at least some of the plurality of grooves 152 may have a uniform groove profile. In some instances, all of the plurality of grooves 152 may have a uniform groove profile. It will be appreciated that in some of the drawings, some of the plurality of grooves 152 may appear to have a non-uniform groove profile, such as a non-uniform groove width, by virtue of viewing angle, looking at a groove as it extends along a curved surface.

In some cases, the elongate shaft 12 is a unitary member, extending proximally from the cutting member 144. In some instances, the elongate shaft 12 may include one or more intermediate sections, such as but not limited to the tapered section 28 and/or the intermediate section 30 disposed between the cutting member 144 and a remainder of the elongate shaft 12. The tapered section 28 and/or the intermediate section 30 may, for example, be integrally milled or otherwise formed with the remainder of the elongate shaft 12. In some cases, the tapered section 28 and/or the intermediate section 30, if present, may be separately formed and secured to the elongate shaft 12. In some cases, the cutting member 144 may be milled into a blank forming the cutting member 104 and the elongate shaft 12 as a unitary structure. In some instances, the cutting member 104 may be separately formed and subsequently secured to the elongate shaft 12.

In some cases, the plurality of grooves 152 may include a number of grooves extending in a helical direction. In some cases, each of the plurality of grooves 152 may extend in a clock-wise direction and/or a counter-clockwise direction. In some instances, the plurality of grooves 152 may include one, two, three, four, five, six or more distinct grooves extending helically in a direction. As an illustrative but non-limiting example, and as shown, the cutting member 144 may include a groove 160, a groove 162, a groove 164 and a groove 166. In some cases, each of the groove 160, the groove 162, the groove 164 and the groove 166 may each extend at least one full revolution about the outer surface 150 of the cutting member 154.

In some cases, a groove may be considered as having a helix angle, which may be defined in terms of an acute angle formed between the particular groove and a line defined by a plane extending parallel to and through the longitudinal axis L. As shown, for example, an acute angle δ₁ may be defined between the groove 162 and the reference line 61 and an acute angle δ₂ may be defined between the groove 164 and the reference line 61. In some cases, as illustrated δ₁ and δ₂ may be different from each other. In some cases, δ₁ and δ₂ may independently range from 30 to 60 degrees. In some cases, δ₁ and δ₂ may each remain constant. In some cases, δ₁ and/or δ₂ may each change as the groove extends helically about the cutting member 144. In some cases, δ₁ and/or δ₂ may each change continuously. In some cases, δ₁ and/or δ₂ may each change in a stepwise fashion.

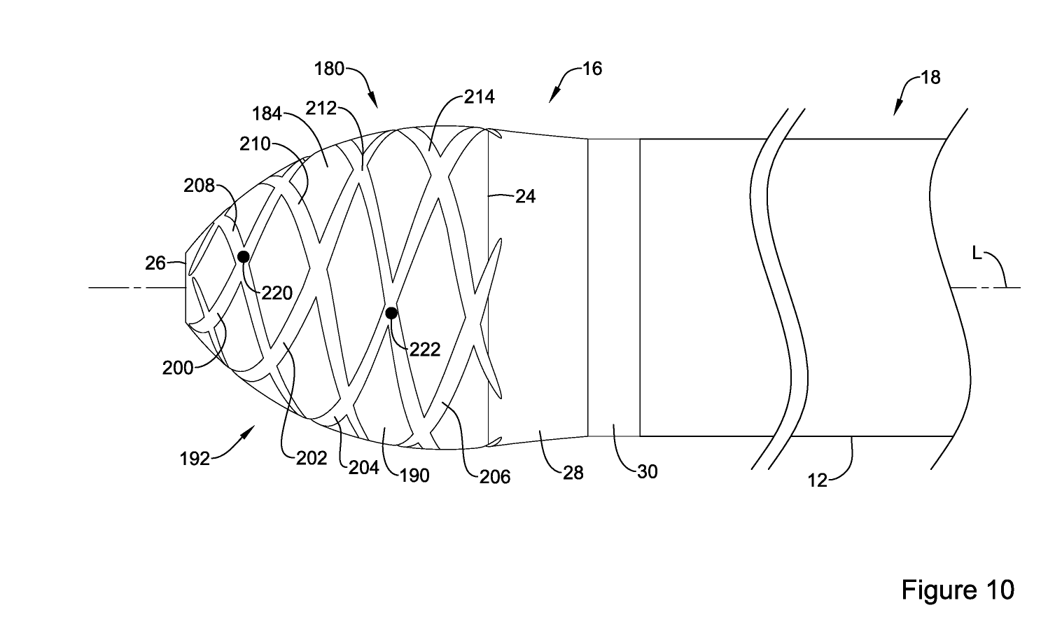

FIG. 10 is a side view of an example atherectomy device 180 that includes grooves extending in a first direction and a second direction, as well having grooves where the angles vary from groove to groove. The atherectomy device 180 includes an elongate shaft 12 and a cutting member 184 that is coupled to the elongate shaft 12. The elongate shaft 12 may be considered as extending from the distal end region 16 to the proximal end region 18. In some cases, as illustrated, the cutting member 184 may be coupled to the distal end region 16 of the elongate shaft 12. The proximal end region 18 is not shown in its entirety, as it will be appreciated that the particular configuration of the proximal end region 18 of the elongate shaft 12 may vary in accordance with the drive mechanism and other features of an atherectomy system with which the atherectomy device 14 may be used.

The cutting member 184 has an outer surface 190 and a plurality of grooves 192 defined within the outer surface 190. In some cases, the outer surface 190 is a curved surface, extending from the proximal end 24 of the cutting member 184 to the distal end 26 of the cutting member 184. In some cases, the outer surface 190 curves (relative to the longitudinal axis L) evenly between the proximal end 24 and the distal end 26. In some instances, the outer surface 190 curves at a varying rate that is related to relative position. In other words, in some cases the outer surface 190 curves at a relatively lower rate closer to the proximal end 24 and curves at a relatively higher rate closer to the distal end 26. In some cases, at least some of the plurality of grooves 192 may have a uniform groove profile. In some instances, all of the plurality of grooves 192 may have a uniform groove profile. It will be appreciated that in some of the drawings, some of the plurality of grooves 192 may appear to have a non-uniform groove profile, such as a non-uniform groove width, by virtue of viewing angle, looking at a groove as it extends along a curved surface.

In some cases, the elongate shaft 12 is a unitary member, extending proximally from the cutting member 184. In some instances, the elongate shaft 12 may include one or more intermediate sections, such as but not limited to the tapered section 28 and/or the intermediate section 30 disposed between the cutting member 144 and a remainder of the elongate shaft 12. The tapered section 28 and/or the intermediate section 30 may, for example, be integrally milled or otherwise formed with the remainder of the elongate shaft 12. In some cases, the tapered section 28 and/or the intermediate section 30, if present, may be separately formed and secured to the elongate shaft 12. In some cases, the cutting member 184 may be milled into a blank forming the cutting member 184 and the elongate shaft 12 as a unitary structure. In some instances, the cutting member 184 may be separately formed and subsequently secured to the elongate shaft 12.

In some cases, the plurality of grooves 192 may include a number of grooves extending in a first helical direction as well as a number of grooves extending in a second helical direction. In some instances, the plurality of grooves 192 may include one, two, three, four, five, six or more distinct grooves extending helically in each direction. As an illustrative but non-limiting example, and as shown, the cutting member 184 may include a groove 200, a groove 202, a groove 204 and a groove 206 each extending in a first helical direction and a groove 208, a groove 210, a groove 212 and a groove 214 extending in a second helical direction.

In some cases, and as a result of groove angles varying from groove to groove, it can be seen that the relative locations of crossing points between grooves varies in accordance with axial position. For example, a crossing point 220 between the groove 200 and the groove 208 can be seen as being vertically above (in the illustrated orientation) the longitudinal axis L. In contrast, a crossing point 222 between the groove 204 and the groove 212 can be seen as being vertically below (in the illustrated orientation) the longitudinal axis.

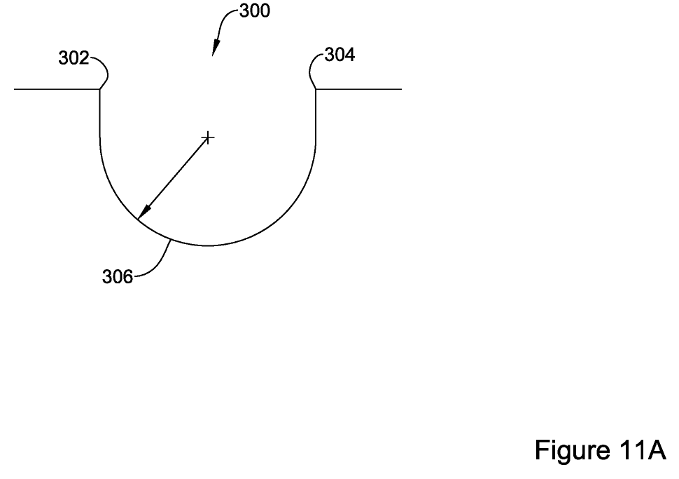

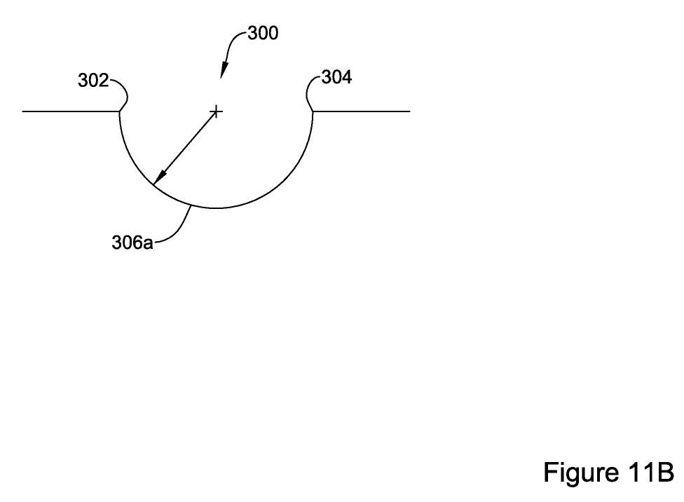

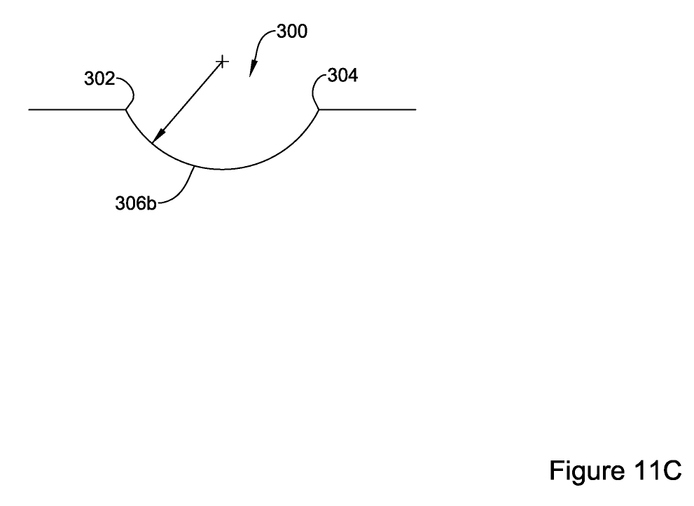

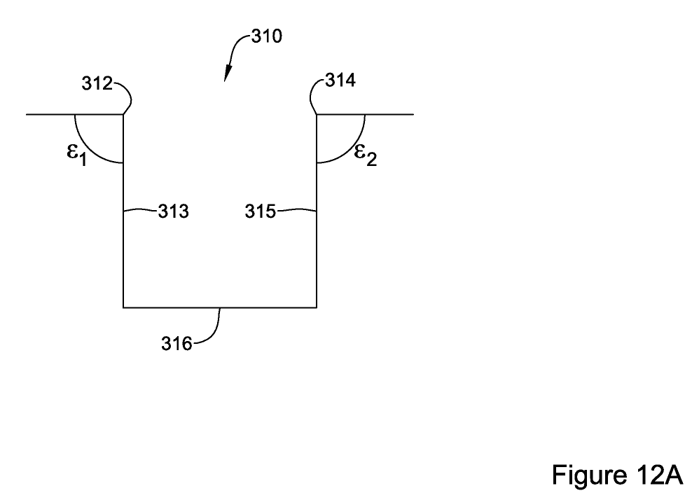

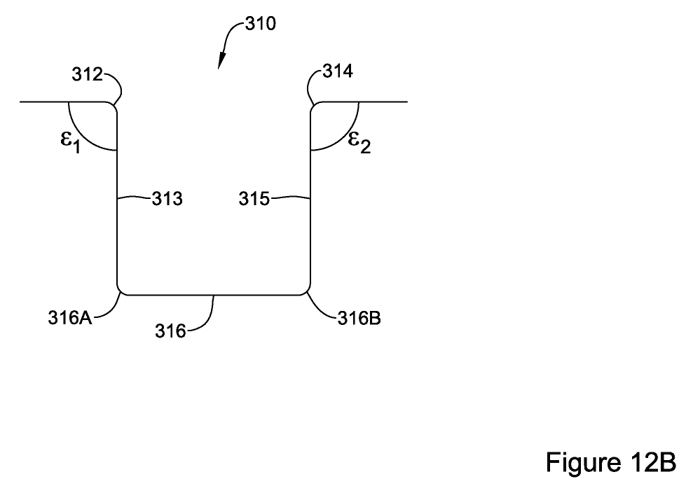

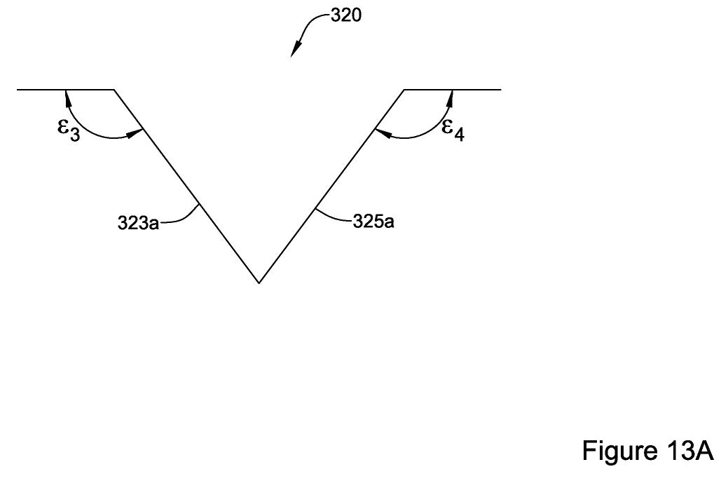

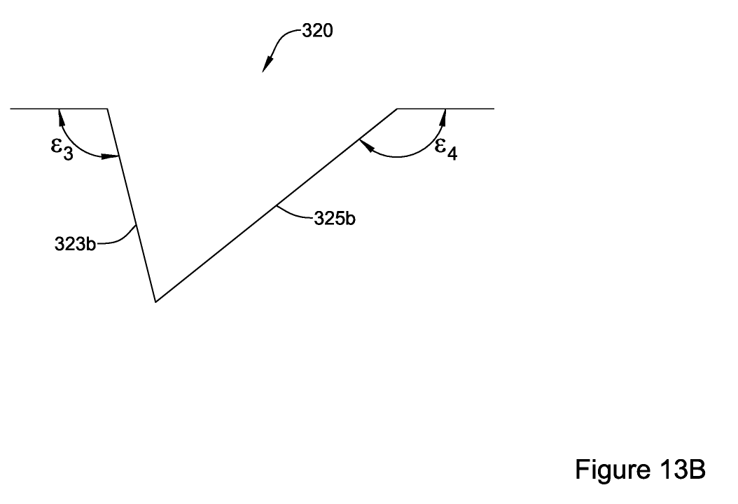

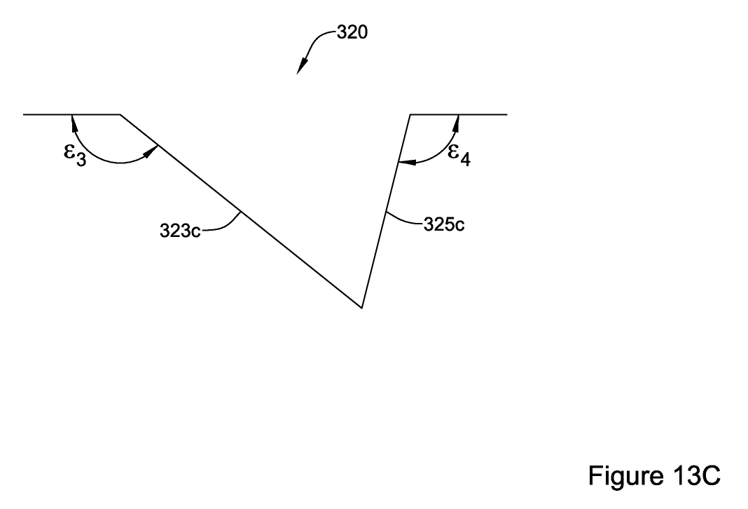

In some cases, the grooves formed in the cutting member 14, the cutting member 64, the cutting member 104, the cutting member 144 and/or the cutting member 184 may take a variety of forms. FIG. 11A, FIG. 11B, FIG. 11C, FIG. 12A, FIG. 12B, FIG. 13A, FIG. 13B and FIG. 13C provide illustrative but non-limiting schematic examples of possible groove configurations. In each FIG., the left side of the groove is considered to be the relatively more distal side while the right side of the groove is considered to be the relatively more proximal side.

FIG. 11A, FIG. 11B and FIG. 11C, for example, each show a schematic diagram of a groove 300 having a leading edge 302 and a trailing edge 304. In most cases, it is the trailing edge 304 that provides the primary cutting surface. The groove 300 has a rounded bottom 306, which may provide advantages in permitting removed material to be carried proximally away from the occlusion. In some cases, the rounded bottom 306 may be considered as having a radius of curvature. In FIG. 11A, the rounded bottom 306 has a center of curvature that is located within the groove 300, below a surface extending between the leading edge 302 and the trailing edge 304. In FIG. 11B, the rounded bottom 306 has a center of curvature that is located on the surface extending between the leading edge 302 and the trailing edge 304. In FIG. 11C, the rounded bottom 306 has a center of curvature that is located above the surface extending between the leading edge 302 and the trailing edge 304.

FIG. 12A is a schematic diagram of a groove 310 having a leading edge 312, a trailing edge 314 and a bottom edge 316. In most cases, it is the trailing edge 314 that provides the primary cutting surface. In some cases, the leading edge 312 defines an angle ε₁ relative to a groove wall 313 while the trailing edge 314 defines an angle ε₂ relative to a groove wall 315. In some cases, ε₁ and ε₂ may each be around 90 degrees. In some cases, as illustrated, the corners defined at the leading edge 312 and the trailing edge 314, as well as those defined between a bottom wall and the groove walls 313 and 315, respectively, may define 90 degree corners. In some cases, as shown for example in FIG. 12B, each of these corners 316A and 316B may be rounded over. In some cases, a groove 310 may include a combination of 90 degree corners and rounded over corners, as desired.

FIG. 13A is a schematic diagram of a groove 320. In some cases, an angle ε₃ may be defined relative to a groove wall 323 while an angle ε₄ may be defined relative to a groove wall 325. In some cases, as shown for example in FIG. 13A, side walls 323a and 325a may define angles ε₃ and ε₄ that are both obtuse angles of 90 to 135 degrees, for example. In some cases, as shown in FIG. 13B, side walls side walls 323b and 325b may define angles ε₃ and ε₄ that are both obtuse angles of 90 to 135 degrees, for example, but where ε₄ is greater than ε₃. In FIG. 13C, for example, side walls side walls 323c and 325c may define angles ε₃ and ε₄ that are both obtuse angles of 90 to 135 degrees, for example, but where ε₃ is greater than ε₄.

In some cases, one or more of the member 14, the cutting member 64, the cutting member 104, the cutting member 144 and/or the cutting member 184 may include an abrasive material applied to an outer surface thereof. In some cases, the grooves themselves may include an abrasive material. In some instances, the grooves may be free of abrasive material. In some cases, the abrasive material may, if present, help to erode hardened or otherwise calcified portions of the occlusion. In some cases, it will be appreciated that the grooves themselves may aid in eroding and/or removing softer material. In some cases, the grooves may also provide for fluid flow to remove excised materials. While the drawings schematically illustrate a cutting member with a relatively small number of grooves, in some cases, a cutting member may have a higher number of grooves. In some cases, a cutting member may for example include 100 or more grooves.

FIG. 14 illustrates an exemplary example of an interventional catheter assembly 410 with which the atherectomy devices described herein may, for example, be used. The interventional catheter assembly 410 includes a console unit 412, a controller 460, and a catheter system 432 having an operating head 440 located at or in proximity to the distal end of the catheter system. The controller 460 may be used to manipulate (e.g. advance and/or rotate) the catheter system 432 and operating head 440, or alternative controls may be provided.

The console unit 412 incorporates an infusion pump 414 and an aspiration pump 416. During operation of the interventional catheter, an infusate conduit 418 draws fluid from an infusate reservoir 420 and operably contacts the infusion pump 414 to provide fluid through an infusion lumen in catheter system 432 to one or more infusion ports provided in proximity to the operating head. Similarly but in reverse, fluids with entrained particulates are withdrawn from the site of intervention through an aspiration lumen in the catheter system 432 and conveyed to an aspiration conduit 422, which is in operable contact with the aspiration pump 416, and communicates with the aspirate collection vessel 424. The console unit 412 may also provide a power source for operating the operating head and system components, or it may be in communication with an external power source. In some cases, the console unit 412 may provide power to the interventional catheter assembly and the controller 460 via a device power port 425 and power cord 426.

Various microprocessor, electronic components, software and firmware components may be provided within or in communication with the console unit for controlling operation of the interventional catheter as described herein. Software may be provided in a machine-readable medium storing executable code and/or other data to provide one or a combination of mechanisms to process user-specific data. Alternatively, various systems and components may be controlled using hardware or firmware implementations. Data storage and processing systems may also be provided in console unit 412. The console unit 412 is generally provided as a reusable assembly and is generally operated outside the sterile field. It may be mountable on a portable stand to facilitate convenient placement during interventions.

One function of the console unit 412 is to provide feedback of system and/or environmental conditions or operating parameters. The console unit may output operational information concerning operating conditions and feedback from the material removal site to the operator. In some cases, the console unit 412 may provide continuously updated output to an operator of operating parameters such as operating head rotation rate, which may include the actual run speed as well as the desired speed; operating head advance rate; aspiration rate and/or volume; infusion rate and/or volume; length of the body or matter to be removed that is traversed; and the like.

Certain automated and selectable control features may be implemented in the console unit 412. Preset routines or programs involving various operating parameters may be preselected, stored and selectable by an operator, for example. Thus, in some cases, the disclosed material removal system implements control features based on an operator's input of specified parameters. Specified parameters may include, for example: lesion length, lesion type and character, such as calcified, fibrotic, lipid/fatty and the like; historical factors, such as restenosis; rate of blood flow; volume of blood flow; percentage of restriction; lumen type and/or location; lumen diameter; desired rotation rate and/or rotation profile for the cutter assembly; desired advance rate and/or advance profile for the cutter assembly; desired aspiration rate and/or profile; desired infusion rate and/or profile; and the like. Based on the specified parameters input by the operator, the control unit may calculate and implement automated operating conditions, such as: cutter assembly rotation rate and profile; cutter assembly advance rate and profile; aspiration rate and profile; infusion rate and profile; cutter assembly size; and the like. Various system operating parameters, operating conditions, patient conditions, and the like may also be recorded and stored during interventions to preserve a record of the patient and intervention operational parameters.

High efficiency aspiration is important in the interventional catheter systems disclosed herein. In certain cases, fluid and associated particulates are aspirated from the intervention site at rates of at least 15 ml/min of operating head run time and, in many cases, fluid and associated particulates are aspirated at rates of at least 25 ml/min of operating head run-time. In exemplary interventional catheter systems, the aspiration site may be more than a meter away from the controller 460 through an aspirate removal passageway located within the catheter system 432 and having a diameter of less than 0.10 inch, for example between about 0.050 to 0.070 inch. The distance that the aspirate travels between controller 460 and console unit 412 may be from about ½ meter to several meters, through an aspirate conduit that is between about 0.125 to about 1.0 inch in diameter. The blood and debris being aspirated are relatively viscous fluids, and achieving a relatively constant and high level of aspiration under these conditions is essential.

In one case, aspiration pump 416 may be a multi-lobed roller pump. The rotation rates of multiple rollers, or of a multi-lobed rotating structure, may be variable or selectable to control the aspiration rate and volume. Roller pumps permit fluid to flow in a conduit through the rollers of the pump at atmospheric pressure, and thus reduce or prevent the formation of bubbles and foam in the liquid being evacuated. Because the aspirate is at atmospheric pressure when it exits the roller pump, a simplified, atmospheric pressure collection vessel may be used rather than an evacuated collection vessel. A simple bag or another collection vessel, such as those used for collection of blood, may be used. For example, a collection bag 424 and a sealed aspiration conduit may be provided as part of a sterile disposable interventional catheter kit. A distal end of the aspiration conduit may be pre-mounted on and sealed to the controller 460. A proximal portion of the aspiration conduit is mounted on the aspiration pump prior to operation of the interventional catheter and the aspirate collection bag is mounted to or in proximity to the control module.

The infusion pump 414 may also be a multi-lobed roller pump employing variable or selectable rotation rates to control the infusion rate and volume. A simple bag or another infusate reservoir, such as those used for intravenous infusions, may be used to supply the infusate. For example, an infusate reservoir 420 having a sealed conduit that is mounted in the infusion pump 416 during operation of the interventional catheter may be provided. In some cases, the sealed infusate conduit may be provided as part of the sterile disposable interventional catheter system and a distal end of the infusate conduit may be pre-mounted on and sealed to the controller 460. A proximal portion of the infusate conduit may be connected to an infusate reservoir, such as a saline bag, and mounted in proximity to the infusion pump prior to operation. A bubble detector 415 may be provided in association with the console unit 412 and the infusate conduit 418 to detect the presence of gas bubbles in the infusate. A control feature that automatically disables the infusion pump and/or power to the operating head may be activated upon detection of a fault (e.g. a bubble) in the infusate conduit.

The console unit 412 may also have control switches for activating and shutting down the aspiration pump and system, and for activating and shutting down the infusion pump and system. These control features may be provided as simple on/off switches. Alternatively, systems providing different levels or rates of aspiration and/or infusion that are selectable by an operator may be provided. In addition, the console unit 412 may be provided with a timing mechanism that determines, and displays, the elapsed time of operation of the operating head and/or the aspiration and infusion systems. The volumes of aspirate withdrawn and the volume of infusate introduced may also be detected and displayed by the console unit 412. Detection systems for monitoring the levels of aspirate and infusate in the respective reservoirs may be incorporated and alarms indicating an overfill condition for the aspirate collection system or a low supply condition for the infusate reservoir may be provided. Back-up aspirate collection and infusate supply systems may also be provided.

In some cases, the console unit 412, together with the aspiration pump 416, the infusion pump 414 and the associated control and display features, may be provided as a separate, re-usable unit, that may be used as standard equipment in operating rooms, for example. In the system illustrated, the console unit 412 is not contaminated by contact with blood or aspirate during operation, and the power and control systems are durable and long-lasting and may be reused for many interventions. The console unit 412 may be provided in a housing designed to sit on a platform during operation, or the housing may be designed for mounting on a portable structure, such as an i.v. pole or another structure. The interventional catheter system, including the catheter system 432 with the operating head 440, the controller 460, the aspirate conduit 422, the aspirate collection vessel 424, and the infusate conduit 418 may be provided as a sterile, single use system kit.

The controller 460, which may be constructed from a durable, sterilizable material, such as hard plastic, may be provided in any convenient ergonomic design and constructed for placement in proximity to and/or in contact with the external body. In one instance, the controller may include an integrated handle for operator convenience in holding and supporting the controller during operation. The catheter system 432, exiting the controller 460, may be axially translatable with respect to the controller 460 as the operating head and catheter system are guided to a target material removal site. It will be appreciated that some of the control and operational features described herein with reference to the controller 460 may be provided in the console unit 412 and, likewise, some of the control and operational features described with reference to the console unit 412 may be provided in the controller 460.

FIG. 15 shows an example rotational atherectomy system 510. The rotational atherectomy system 510 may include a rotational atherectomy device 512 and a controller 514 for controlling the rotational atherectomy device 512. The rotational atherectomy device 512 may include a housing 516 and an elongate shaft 518 extending distally from the housing 516 to a cutting member 520 located at a distal end of the elongate shaft 518. The elongate shaft 518 may include a drive shaft 524 to provide rotational motion to the cutting member 520. In some instances, the elongate shaft 518 may include an outer tubular member 522 having a lumen extending therethrough and the drive shaft 524 may extend through the lumen of the outer tubular member 522. The drive shaft 524, which may be fixed to the cutting member 520, may be rotatable relative to the outer tubular member 522 to rotate the cutting member 520. In some instances the axial position of the cutting member 520 relative to the outer tubular member 522 may be adjusted by moving the drive shaft 524 longitudinally relative to the outer tubular member 522. For example, the atherectomy device 512 may include an advancer assembly 526 positioned in the housing 516, or otherwise provided with the housing 516, that is longitudinally movable relative to the housing 516. The outer tubular member 522 may be coupled to the housing 516 while the drive shaft 524 may be coupled to the advancer assembly 526. Accordingly, the drive shaft 524 (and thus the cutting member 520) may be longitudinally movable relative to the outer tubular member 522 by actuating the advancer assembly 526 relative to the housing 516.

The rotational atherectomy device 512 may include a prime mover (not shown) to provide rotational motion to the drive shaft 524 to rotate the cutting member 520. For example, in some instances the prime mover may be a fluid turbine within the housing 516, such as provided with the advancer assembly 526. In other instances, however, the prime mover may be an electrical motor, or the like. The controller 514 may be used to control the prime mover. For example, the user may provide power to the prime mover and/or control the speed of rotation of the drive shaft 524 via the controller 514. For example, the front panel 528 of the controller 514 may include a user interface including a power switch, speed control mechanism (e.g., a speed control knob and/or buttons), a display, and/or other features for controlling the rotational atherectomy device 512. In some instances, the rotational atherectomy system 510 may include a remote control device 530, such as a foot pedal, a hand control, or other mechanism which may be used to control the power and/or speed to the prime mover, for example.

In instances in which the prime mover is a turbine, the rotational atherectomy system 510 may also include a pressurized fluid source 532 providing a pressurized fluid to the turbine to rotate the drive shaft 524. In some instances, as shown, the pressurized fluid source 532 may be a tank of pressurized fluid (e.g., compressed air), which may or may not include an air compressor. In other instances, the pressured fluid source 532 may be provided external of the rotational atherectomy system 510, such as from a wall outlet at the medical facility. The pressured fluid source 532 may be coupled to the controller 514 via a fluid conduit 534, which in turn is coupled to the rotational atherectomy device 512 via a fluid conduit 536. The controller 514 may regulate the flow and/or pressure of fluid through the fluid conduit 536 to the rotational atherectomy device 512 to control the speed of rotation of the drive shaft 524 and cutting member 520, for instance.

In instances in which the prime mover is an electric motor, the electric motor may be coupled to the controller 514 via an electrical connection to control the electric motor and/or provide electricity to the electric motor.

In some instances, the rotational atherectomy device 512 may include a speed sensor, such as an optical speed sensor, coupled to the controller 514 via a connector 538, such as a fiber optic connector to provide speed data to the controller 514. In other instances, an electronic sensor, such as a Hall Effect sensor, or other type of sensor may be used to sense the speed of the drive shaft 524 and cutting member 520. The speed data may be displayed, such as on the front panel 528 and/or the controller 514, and/or used to control the speed of the cutting member 520, such as maintaining a desired speed of the cutting member 520 during a medical procedure.

In some instances, the rotational atherectomy system 510 may be configured to infuse fluid through the elongate shaft 518 to the treatment site and/or aspirate fluid through the elongate shaft 518 from the treatment site. For example, the rotational atherectomy system 510 may include a fluid supply 540 for providing a flow of fluid through a lumen of the elongate shaft 518 to a treatment site. In some instances the fluid supply 540 may include a saline bag 542 which may be pressurized by a pressure cuff 544 to provide a pressurized fluid (e.g., saline) to the rotational atherectomy device 512 through a fluid supply line 546. In other instances, an infusion pump, such as a peristaltic pump, may be used to deliver pressurized fluid to the rotational atherectomy device 512. Additionally or alternatively, in some cases the rotational atherectomy system 510 may be configured to aspirate fluid from the treatment site. For example, the rotational atherectomy system 510 may include an aspiration pump, such as a peristaltic pump, to generate a vacuum to aspirate fluid through a lumen of the elongate shaft 518 to a fluid collection container (not shown), if desired.

In some instances, the elongate shaft 518 of the rotational atherectomy device 512 may be advanced over a guidewire 548 to a treatment site. For example, the drive shaft 524 may include a guidewire lumen through which the guidewire 548 may pass. Additionally or alternatively, the elongate shaft 518 may be advanced through a lumen of a guide catheter to a treatment site.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An atherectomy device, comprising:
an elongate shaft having a proximal end region and a distal end region;
a cutting member positioned adjacent the distal end region of the elongate shaft, the cutting member extending along a longitudinal axis and having an outer surface; and
a plurality of helical grooves defined in the outer surface of the cutting member, at least two of the plurality of helical grooves extending at least one full helical revolution around the longitudinal axis.

2. The device of claim 1, wherein the plurality of helical grooves includes at least a first groove extending in a first helical direction around the longitudinal axis, and at least a second groove extending in a second helical direction around the longitudinal axis, wherein the first direction is different from the second direction, such that the first groove and the second groove intersect each other at least once.

3. The device of claim 1, wherein the plurality of helical grooves includes first and second grooves extending in a first helical direction around the cutting member.

4. The device of claim 1, wherein at least two of the plurality of grooves are parallel to one another.

5. The device of claim 1, wherein a helix angle of at least one of the plurality of helical grooves changes as the at least one helical groove extends around the longitudinal axis.

6. The device of claim 1, wherein the plurality of helical grooves have a helix angle in the range of 30 to 60 degrees.

7. The device of claim 1, wherein the plurality of helical grooves includes three or more helical grooves.

8. The device of claim 1, further including an abrasive material disposed on the outer surface of the cutting member.

9. The device of claim 1, wherein the plurality of grooves are spaced equally around a central longitudinal axis of the cutting member.

10. An atherectomy device, comprising:
an elongate shaft having a proximal end region and a distal end region;
a cutting member positioned adjacent the distal end region of the elongate shaft, the cutting member extending along a longitudinal axis and having an outer surface; and
a plurality of helical grooves including at least a first helical groove extending in a first helical direction around the longitudinal axis, and at least a second helical groove extending in a second helical direction around the longitudinal axis, wherein the first direction is different from the second direction, such that the first helical groove and the second helical groove intersect each other at least once.

11. The device of claim 10, further including one or more additional grooves extending in the first helical direction.

12. The device of claim 10, further including one or more additional grooves extending in the second helical direction.

13. The device of claim 10, wherein a helix angle of at least one of the plurality of helical grooves changes as the at least one helical groove extends around the longitudinal axis.

14. The device of claim 10, wherein the plurality of helical grooves have a helix angle in the range of 30 to 60 degrees.

15. The device of claim 10, wherein the plurality of helical grooves includes three or more helical grooves.

16. The device of claim 10, further including an abrasive material disposed on the outer surface of the cutting member.

17. The device of claim 10, wherein the plurality of grooves includes two or more helical grooves extending in the first helical direction around the longitudinal axis, and two or more helical grooves extending in the second helical direction around the longitudinal axis.

18. The device of claim 17, wherein the two or more helical grooves extending in the first helical direction around the longitudinal axis are spaced equally around a central longitudinal axis of the cutting member.

19. The device of claim 17, wherein the two or more helical grooves extending in the second helical direction around the longitudinal axis are spaced equally around a central longitudinal axis of the cutting member.

20. An atherectomy device, comprising:
an elongate shaft having a proximal end region and a distal end region;
a cutting member positioned adjacent the distal end region of the elongate shaft, the cutting member extending along a longitudinal axis and having an outer surface; and
a plurality of helical grooves including at least a first helical groove extending in a first helical direction around the longitudinal axis, and at least a second helical groove extending in a second helical direction around the longitudinal axis, wherein the first direction is different from the second direction, such that the first helical groove and the second helical groove intersect each other at least once; and
wherein the at least the first helical groove and the at least a second helical groove each extend at least one full helical revolution around the longitudinal axis.
